(19) United States
(12) Patent Application Publication
(10) Pub. No.: US 2023/0145721 A1
(43) Pub. Date: May 11, 2023

(54) AIR PURIFICATION DEVICE

(71) Applicant: Medify Air, LLC, Deerfield Beach, FL (US)

(72) Inventor: J Henry SCOTT, Deerfield Beach, FL (US)

(21) Appl. No.: 17/524,287

(22) Filed: Nov. 11, 2021

(51) Int. Cl. B01D 46/00; A61L 9/20

(52) U.S. Cl. CPC B01D 46/0028; B01D 46/0049; A61L 9/20; B01D 2273/30; B01D 2279/65; A61L 2209/14

(57) ABSTRACT

A device for purifying air using UV light. In one embodiment, a device includes: a housing, the housing at least partially defining an air flow path and an inner chamber, the air flow path including at least one linear air flow segment and at least one non-linear air flow segment; a UV chamber within the inner chamber, the UV chamber at least partially defining the at least one non-linear air flow segment; a plurality of structural features within the UV chamber, each of the plurality of structural features creating at least one transition area, each of the at least one transition area being configured to change a direction of an air flow within the at least one non-linear air flow segment; and at least one UV bulb, each of the at least one UV bulb being positioned proximate a one of the at least one transition area.

### CROSS-REFERENCE TO RELATED APPLICATIONS

N/A.

### GOVERNMENT RIGHTS STATEMNT

N/A.

### FIELD

The present technology is related generally to a device for the purification of air using ultraviolet (UV) light.

### BACKGROUND

Environmental contaminants can adversely affect the health of humans and animals who inhale the contaminated air. For example, contaminants such as airborne viruses, bacteria, molds, and other microorganisms and chemical pollutants, can cause eye, nose, and throat irritation, headache, fatigue, dizziness, breathing difficulties, asthma attacks, pathogen-related disease, and other symptoms and effects. The effects caused by inhalation of contaminated air can be particularly harmful in enclosed indoor spaces.

Therefore, providing a means for decontaminating or purifying indoor air in buildings (for example, commercial, residential, and industrial buildings) is important to improve indoor air quality. Many air purification devices for removal of pollutants from indoor air have been used, such as central heating, ventilating, and air-conditioning (HVAC) filters, high-efficiency particular air (HEPA) filters, photo-electrochemical oxidation (PECO) filters, ultraviolet (UV) light, ionization systems, and others. However, traditional filters such as HVAC and HEPA filters only trap, and do not neutralize or kill, airborne contaminants. Likewise, some other filters may not be effective in reducing the overall particulate concentration in the air. Although UV light may neutralize or kill contaminants such as bacteria, viruses, and molds, the contaminated air must be exposed to the UV light for an adequate period of time. Most UV air purification devices provide an inadequate exposure time between the UV light and air passing therethrough, making them largely ineffective at air purification.

### SUMMARY

Some embodiments advantageously provide a device for purifying air using UV light. In one embodiment, a device includes: an air flow path, the air flow path including at least one linear air flow segment and at least one non-linear air flow segment; a UV chamber, the UV chamber at least partially defining the at least one non-linear air flow segment; and at least one UV bulb within the UV chamber.

In some aspects of the embodiment, the device further includes at least one structural feature within the UV chamber. In some aspects of the embodiment, the device further includes a housing, the housing defining an inner chamber, the UV chamber being within the inner chamber.

In some aspects of the embodiment, the device further includes a UV chamber frame, the UV chamber frame including the at least one structure feature.

In some aspects of the embodiment, the at least one structural feature is a protrusion that extends into the UV chamber. In some aspects of the embodiment, each of the at least one protrusion creates a transition area configured to change a direction of an air flow. In some aspects of the embodiment, each of the at least one UV bulb is located proximate a transition area.

In some aspects of the embodiment, each of the at least one structural feature creates a transition area configured to change a direction of an air flow; and each of the at least one UV bulb is coupled to a surface of the UV chamber frame. In some aspects of the embodiment, each of the at least one UV bulb is coupled to a surface of the UV chamber frame at a location proximate the transition area. In some aspects of the embodiment, each of the at least one UV bulb is elongate and extends from the UV chamber frame and into the UV chamber.

In some aspects of the embodiment, each of the at least one UV bulb emits UV-C light.

In some aspects of the embodiment, the at least one non-linear air flow segment includes at least two non-linear air flow segments.

In some aspects of the embodiment, the device further includes: at least one fan configured to draw air through the air flow path; and at least one filter positioned between the UV chamber and the fan.

In one embodiment a device includes: a housing, the housing at least partially defining an air flow path and an inner chamber, the air flow path including at least one linear air flow segment and at least one non-linear air flow segment; a UV chamber within the inner chamber, the UV chamber at least partially defining the at least one non-linear air flow segment; a plurality of structural features within the UV chamber, each of the plurality of structural features creating at least one transition area, each of the at least one transition area being configured to change a direction of an air flow within the at least one non-linear air flow segment; and at least one UV bulb, each of the at least one UV bulb being positioned proximate a one of the at least one transition area.

In some aspects of the embodiment, the device further includes a UV chamber frame within the UV chamber, each of the at least one UV bulb being mounted to the UV chamber frame. In some aspects of the embodiment, each of the plurality of structural features is a protrusion extending from the UV chamber frame.

In some aspects of the embodiment, the at least one non-linear air flow segment includes two non-linear air flow segments.

In some aspects of the embodiment, the housing includes a first end and a second end opposite the first end, the first end defining a plurality of air inlets, the air flow path extending from the first end to the second end.

In some aspects of the embodiment, the device further includes: a fan configured to draw air through the air flow path, the fan being within the second end of the housing; and at least one filter positioned between the UV chamber and the fan.

In one embodiment, an air purification device includes: a housing, the housing at least partially defining an air flow path and an inner chamber, the housing including a first end and a second end opposite the first end, the first end having at least one air inlet and the second end having at least one air outlet, the air flow path extending from the first end of the housing to the second end of the housing and including at least one linear air flow segment and at least one non-linear air flow segment; a UV chamber within the inner chamber, the UV chamber at least partially defining the at least one non-linear air flow segment; a UV chamber frame within the UV chamber, the UV chamber frame including a plurality of protrusions, each of the plurality of protrusions creating at least one transition area configured to change a direction and to reduce a speed of an air flow within the at least one non-linear air flow segment; at least one UV bulb, each of the at least one UV bulb being positioned proximate a one of the at least one transition area; fan within the second end of the housing, the fan being configured to drawn air through the air flow path; and at least one filter between the UV chamber and the fan.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 shows a cross-sectional vies of an exemplary air purification device, in accordance with the present disclosure.

### DETAILED DESCRIPTION

Before describing in detail exemplary embodiments, it is noted that the embodiments reside primarily in combinations of apparatus components and steps related to air purification using UV light. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

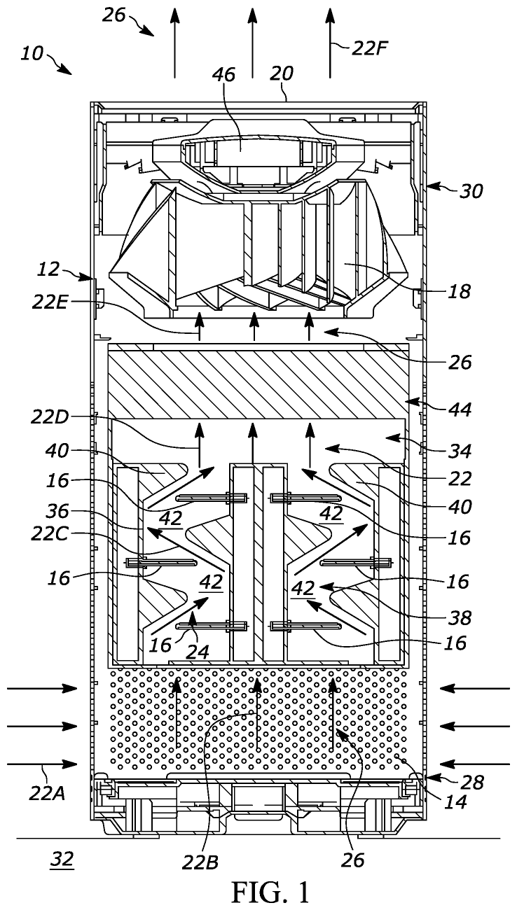

Referring now to FIG. 1, a cross-sectional view of an exemplary air purification device is shown. In general, the air purification device 10 generally includes a housing 12, at least one air inlet 14, at least one UV light bulb 16, a fan 18, and at least one air outlet 20. The housing, or components thereof, at least partially defines an air flow path 22, at least a portion of the air flow path 22 including a non-linear air flow segment 24. Additionally, in one embodiment at least a portion of the air flow path includes a linear air flow segment 26. Although referred to herein as a “linear air flow segment,” it will be understood that the air flow path of the linear air flow segment may be linear, at least partially linear, or more linear than in the non-linear air flow segment 24. That is, the linear air flow segment 26 is not deliberately configured to slow the flow rate of air. As used herein, the term “purification” refers to the treatment of air to neutralize or kill airborne contaminants, such as bacteria, viruses, molds, and/or other microorganisms, pieces or fragments thereof, and/or other airborne particulate matter, and to remove such contaminants from the air and/or render them harmless or ineffective against humans, pets, or other organisms.

In one embodiment, the housing 12 is composed of at least one rigid and/or semi-rigid material, such as plastic, metal, or wood. In the embodiment shown in FIG. 1, the housing has a substantially rectangular shape, but it will be understood that suitable shapes, sizes, and/or configurations other than those shown may be used. In one embodiment, the housing 12 includes a first end 28 and a second end 30 opposite the first end 28. In some embodiments, the air purification device 10 is configured to be placed on a horizontal or at least substantially horizontal surface, such as a floor or table top (referred to herein as a mounting surface 32), with the first end 28 on or in contact with the mounting surface 32 and the second end 30 extending away from the mounting surface 32, as shown in FIG. 1. However, in other embodiments, the air purification device 10 may be configured to be mounted to a vertical or at least substantially vertical mounting surface, such as a wall, post, or ramp, or suspended from a structure such as a beam, pole, pipe, ceiling, or the like.

In one embodiment, air is drawn into the at least one air inlet 14, which in one embodiment are located at or proximate the first end 28. For example, the at least one air inlet 14 may include a plurality of apertures extending through the housing 12 and into an inner chamber 34 at least partially defined by the housing 12, as indicated in FIG. 1 by arrows 22A. In one embodiment, the air flow path indicated by arrows 22B is a linear air flow segment 26. Although each of the apertures is shown in FIG. 1 as having a circular shape, it will be understood that other suitable shapes, sizes, and/or configurations of the at least one air inlet 14 may be used.

In one embodiment, air is then drawn (for example, upward) from the inner chamber 34 into the non-linear air flow segment 24 by the fan 18, as indicated in FIG. 1 by arrows 22B. In one embodiment, the air flow path indicated by arrows 22B is a linear air flow segment 26. In one embodiment, the air purification device 10 further includes a UV chamber frame 36 mounted, affixed, and/or integrated with the housing 12 within the inner chamber 34. The UV chamber frame 36 at least partially defines a UV chamber 38, through which the non-linear air flow segment 24 passes, as indicated in FIG. 1 by arrows 22C. The UV chamber frame 36 may be composed of rigid, semi-rigid, flexible, resiliently deflectable, and/or other materials that are the same and/or different than those from which the housing 12 is composed. In some embodiments, the UV chamber frame 36 is manufactured as a separate piece or pieces that are mounted or affixed within the inner chamber 34 of the housing 12. In other embodiments, the UV chamber frame 36 is integrated with or manufactured as a single unitary piece with the housing 12.

The UV chamber 38 includes one or more protuberances, indentations, fins, ribs, fingers, bumps, ridges, or other structural features 40 that are configured to disrupt a linear air flow into a non-linear air flow. Thus, the other structural features 40 at least partially create the non-linear air flow segment 24 and cause air to flow in a non-linear path, such as one or more tortuous, serpentine, curved, helical, wavy, turbulent, and/or double-back segments. Further, in some embodiments, such as that shown in FIG. 1, the UV chamber 38 is configured to divide the air flow into two or more non-linear air flow segments 24.

The other structural features 40 create a plurality of transition areas 42 where the direction of air flow changes, such as bends, curves, redirects, or the like, as indicated in FIG. 1 by arrows 22C. In one embodiment, at least one UV light bulb 16 is mounted to the UV chamber frame 36 at or proximate each (or at least one) transition area 42, or at a location where the air at each transition areas 42 is exposed to UV light emitted by each UV light bulb 16. In one non-limiting example, as shown in FIG. 1, each at least one UV light bulb 16 is mounted or affixed to a wall of the UV chamber frame 36 that is opposite a transition area 42. Further, in one embodiment, each at least one UV light bulb 16 is elongate (for example, tube shaped) and extends from the wall of the UV chamber frame 36 into the UV chamber 38. The configuration of the UV chamber 38, such as the transition areas 42, slow the flow rate of air passing therethrough (within the non-linear air flow segment 24) to increase the time the air is exposed to the UV light emitted by the at least one UV light bulb 16. In some embodiments, the flow rate of air may be slowed enough to allow for more than three times the UV exposure of currently known UV air purification devices. Additionally, the flow rate of air may be slowest at, within, or immediately adjacent the transition areas 42, so air at these locations has an even greater exposure to UV light because of the location of the UV light bulbs 16. Greater UV exposure increases the efficiency of the UV light (that is, the amount of biological particles, such as bacteria, viruses, and/or molds, that are neutralized or killed by the UV light). This, in turn, allows the air purification device 10 to more efficiently purify the air passing therethrough. For example, the air purification device 10 may be capable of purifying a larger volume of air and/or purifying air more rapidly than currently known air purification devices of similar size and/or power.

In one embodiment, the at least one UV light bulb 16 emits UV-C light. Further, in one embodiment, the walls of the UV chamber frame 36 that define the UV chamber 38 are at least partially coated with a UV-reflective coating or embedded with UV-reflective materials. In one embodiment, the UV-reflective coating and/or materials provide up to 95% reflectivity. In one embodiment, the UV-reflective coating and/or materials provide at least approximately 95% reflectivity. In some embodiments, each of the at least one UV light bulb 16 may have a size, shape, and/or configuration that is different than that shown. Additionally, more or fewer UV light bulbs 16 than shown may be used, and at different locations. In one non-limiting example, the air purification device 10 may further include additional UV light bulbs on and/or proximate at least one structural feature 40 that extends into the UV chamber 38, such as UV light bulb(s) that are embedded in, flush with, or that themselves do not extend into the UV chamber 38 as far as the at least one UV light bulb 16 shown in FIG. 1. Such a configuration may provide additional UV exposure to the air passing through the UV chamber 38.

In one embodiment, the air is then drawn (for example, upward) from the UV chamber 38 and non-linear air flow segment 24 by the fan 18 and through a filter 44, as indicated in FIG. 1 by arrows 22D. In one embodiment, the air flow path indicated by arrows 22B is a linear air flow segment 26. In one embodiment, the filter 44 is a HEPA filter (for example, HEPA H13-14) that provides capture of 99.9%-99.99% of contaminant remains and other particles. In one embodiment, the filter 44 is located downstream of the UV chamber 38. However, it will be understood that additional configurations may be used. For example, the filter 44 may be located upstream of the UV chamber 38, or the air purification device 10 may include both a filter downstream of the UV chamber 38 and a filter upstream of the UV chamber 38. Further, any number of filters may be used.

In one embodiment, the air is then drawn (for example, upward) from the filter 44 and through the fan 18, as indicated in FIG. 1 by arrows 22E. In one embodiment, the air flow path indicated by arrows 22B is a linear air flow segment 26. In one embodiment, the fan 18 creates and maintains a steady flow of air through the air purification device 10, and at an air flow rate that ensures the air flows through the UV chamber 38 slowly enough to allow for adequate UV exposure. A target air flow rate may depend on factors such as the dimensions of the area to be treated (for example, room size), the model and size of the device, and others. However, in one non-limiting example, the fan 18 is configured to draw air into the air purification device 10 at an air flow rate of between approximately 100 ft³/min and approximately 1000 ft³/min. In one embodiment, the fan 18 includes a DC, low-speed, high-torque motor 46, and the motor 46 and fan 18 are configured to operate at low noise levels while ensuring sufficient air flow through the air purification device 10.

In one embodiment, the air then passes from the fan 18 and is expelled through the at least one air outlet 20, as indicated in FIG. 1 by arrows 22F. In one embodiment, the at least one air outlet 20 is located at or proximate the second end 30. For example, the at least one air outlet 20 may include a plurality of apertures extending through the housing 12 and into the inner chamber 34, such as a portion of the inner chamber 34 that at least partially houses the fan 18.

In one embodiment, a device 10 includes: an air flow path 22, the air flow path 22 including at least one linear air flow segment 26 and at least one non-linear air flow segment 24; a UV chamber 38, the UV chamber 38 at least partially defining the at least one non-linear air flow segment 24; and at least one UV bulb 16 within the UV chamber 38.

In some aspects of the embodiment, the device 10 further includes at least one structural feature 40 within the UV chamber 38. In some aspects of the embodiment, the device 10 further includes a housing 12, the housing 12 defining an inner chamber 34, the UV chamber 38 being within the inner chamber 34.

In some aspects of the embodiment, the device 10 further includes a UV chamber frame 36, the UV chamber frame 36 including the at least one structure feature 40.

In some aspects of the embodiment, the at least one structural feature 40 is a protrusion that extends into the UV chamber 38. In some aspects of the embodiment, each of the at least one protrusion creates a transition area 42 configured to change a direction of an air flow. In some aspects of the embodiment, each of the at least one UV bulb 16 is located proximate a transition area 42.

In some aspects of the embodiment, each of the at least one structural feature 40 creates a transition area 42 configured to change a direction of an air flow; and each of the at least one UV bulb 16 is coupled to a surface of the UV chamber frame 36. In some aspects of the embodiment, each of the at least one UV bulb 16 is coupled to a surface of the UV chamber frame 36 at a location proximate the transition area 42. In some aspects of the embodiment, each of the at least one UV bulb 16 is elongate and extends from the UV chamber frame 36 and into the UV chamber 34.

In some aspects of the embodiment, each of the at least one UV bulb 16 emits UV-C light.

In some aspects of the embodiment, the at least one non-linear air flow segment 24 includes at least two non-linear air flow segments 24.

In some aspects of the embodiment, the device 10 further includes: at least one fan 18 configured to draw air through the air flow path 22; and at least one filter 44 positioned between the UV chamber 34 and the fan 18.

In one embodiment a device 10 includes: a housing 12, the housing 12 at least partially defining an air flow path 22 and an inner chamber 34, the air flow path 22 including at least one linear air flow segment 26 and at least one non-linear air flow segment 24; a UV chamber 38 within the inner chamber 34, the UV chamber 38 at least partially defining the at least one non-linear air flow segment 24; a plurality of structural features 40 within the UV chamber 38, each of the plurality of structural features 40 creating at least one transition area 42, each of the at least one transition area 42 being configured to change a direction of an air flow within the at least one non-linear air flow segment 24; and at least one UV bulb 16, each of the at least one UV bulb 16 being positioned proximate a one of the at least one transition area 42.

In some aspects of the embodiment, the device 10 further includes a UV chamber frame 36 within the UV chamber 34, each of the at least one UV bulb 16 being mounted to the UV chamber frame 36. In some aspects of the embodiment, each of the plurality of structural features 40 is a protrusion extending from the UV chamber frame 36.

In some aspects of the embodiment, the at least one non-linear air flow segment 24 includes two non-linear air flow segments 24.

In some aspects of the embodiment, the housing 12 includes a first end 28 and a second end 30 opposite the first end 28, the first end 28 defining a plurality of air inlets 14, the air flow path 22 extending from the first end 28 to the second end 30. In some aspects of the embodiment, the device 10 further includes: a fan 18 configured to draw air through the air flow path 22, the fan 18 being within the second end 30 of the housing 12; and at least one filter 44 positioned between the UV chamber 38 and the fan 18.

In one embodiment, an air purification device 10 includes: a housing 12, the housing 12 at least partially defining an air flow path 22 and an inner chamber 34, the housing 12 including a first end 28 and a second end 30 opposite the first end 28, the first end 28 having at least one air inlet 14 and the second end 30 having at least one air outlet 20, the air flow path 22 extending from the first end 28 of the housing 12 to the second end 30 of the housing 12 and including at least one linear air flow segment 26 and at least one non-linear air flow segment 24; a UV chamber 38 within the inner chamber 34, the UV chamber 38 at least partially defining the at least one non-linear air flow segment 24; a UV chamber frame 36 within the UV chamber 38, the UV chamber frame 36 including a plurality of protrusions 40, each of the plurality of protrusions 40 creating at least one transition area 42 configured to change a direction and to reduce a speed of an air flow within the at least one non-linear air flow segment 24; at least one UV bulb 16, each of the at least one UV bulb 16 being positioned proximate a one of the at least one transition area 42; fan 18 within the second end 30 of the housing 12, the fan 18 being configured to drawn air through the air flow path 22; and at least one filter 44 between the UV chamber 38 and the fan 18.

It should be understood that various aspects disclosed herein may be combined in different combinations than the combinations specifically presented int eh description and the accompanying drawings.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

What is claimed is:

1. A device, the device comprising:
an air flow path, the air flow path including at least one linear air flow segment and at least one non-linear air flow segment;
a UV chamber, the UV chamber at least partially defining the at least one non-linear air flow segment; and
at least one UV bulb within the UV chamber.

2. The device of claim 1, further comprising at least one structural feature within the UV chamber.

3. The device of claim 2, further comprising a housing, the housing defining an inner chamber, the UV chamber being within the inner chamber.

4. The device of claim 3, further comprising a UV chamber frame, the UV chamber frame including the at least one structure feature.

5. The device of claim 2, wherein the at least one structural feature is a protrusion that extends into the UV chamber.

6. The device of claim 5, wherein each of the at least one protrusion creates a transition area configured to change a direction of an air flow.

7. The device of claim 6, wherein each of the at least one UV bulb is located proximate a transition area.

8. The device of claim 4, wherein:
each of the at least one structural feature creates a transition area configured to change a direction of an air flow; and
each of the at least one UV bulb is coupled to a surface of the UV chamber frame.

9. The device of claim 8, wherein each of the at least one UV bulb is coupled to a surface of the UV chamber frame at a location proximate the transition area.

10. The device of claim 9, wherein each of the at least one UV bulb is elongate and extends from the UV chamber frame and into the UV chamber.

11. The device of claim 1, wherein each of the at least one UV bulb emits UV-C light.

12. The device of claim 1, wherein the at least one non-linear air flow segment includes at least two non-linear air flow segments.

13. The device of claim 1, further comprising:
at least one fan configured to draw air through the air flow path; and
at least one filter positioned between the UV chamber and the fan.

14. A device, the device comprising:
a housing, the housing at least partially defining an air flow path and an inner chamber, the air flow path including at least one linear air flow segment and at least one non-linear air flow segment;
a UV chamber within the inner chamber, the UV chamber at least partially defining the at least one non-linear air flow segment;
a plurality of structural features within the UV chamber, each of the plurality of structural features creating at least one transition area, each of the at least one transition area being configured to change a direction of an air flow within the at least one non-linear air flow segment; and
at least one UV bulb, each of the at least one UV bulb being positioned proximate a one of the at least one transition area.

15. The device of claim 14, further comprising a UV chamber frame within the UV chamber, each of the at least one UV bulb being mounted to the UV chamber frame.

16. The device of claim 15, wherein each of the plurality of structural features is a protrusion extending from the UV chamber frame.

17. The device of claim 14, wherein the at least one non-linear air flow segment includes two non-linear air flow segments.

18. The device of claim 14, wherein the housing includes a first end and a second end opposite the first end, the first end defining a plurality of air inlets, the air flow path extending from the first end to the second end.

19. The device of claim 18, further comprising:
a fan configured to draw air through the air flow path, the fan being within the second end of the housing; and
at least one filter positioned between the UV chamber and the fan.

20. An air purification device, the air purification device comprising:
a housing, the housing at least partially defining an air flow path and an inner chamber, the housing including a first end and a second end opposite the first end, the first end having at least one air inlet and the second end having at least one air outlet, the air flow path extending from the first end of the housing to the second end of the housing and including at least one linear air flow segment and at least one non-linear air flow segment;
a UV chamber within the inner chamber, the UV chamber at least partially defining the at least one non-linear air flow segment;
a UV chamber frame within the UV chamber, the UV chamber frame including a plurality of protrusions, each of the plurality of protrusions creating at least one transition area configured to change a direction and to reduce a speed of an air flow within the at least one non-linear air flow segment;
at least one UV bulb, each of the at least one UV bulb being positioned proximate a one of the at least one transition area;
fan within the second end of the housing, the fan being configured to drawn air through the air flow path; and
at least one filter between the UV chamber and the fan.
